(19) United States
(12) Patent Application Publication
(10) Pub. No.: US 2022/0257264 A1
(43) Pub. Date: Aug. 18, 2022

(54) JIG FOR KNEE REVISION SURGERY

(71) Applicant: ORTHOSOFT ULC, Montreal (CA)

(72) Inventors: Pierre COUTURE, Montreal (CA); Louis-Philippe AMIOT, Montreal (CA); Maxime RIOUX, Montreal (CA)

(21) Appl. No.: 17/673,257

(22) Filed: Feb. 16, 2022

Related U.S. Application Data
(60) Provisional application No. 63/149,840, filed on Feb. 16, 2021.

(51) Int. Cl. A61B 17/15; A61F 2/46; A61B 34/10; A61F 2/38

(52) U.S. Cl. CPC A61B 17/155; A61F 2/461; A61F 2002/3069; A61F 2/3859; A61B 34/10

(57) ABSTRACT

A jig for revision surgery includes a body defining a contact surface(s) negatively corresponding to an articular surface of a primary implant. The body is configured to be coupled in a unique complementary coupling via engagement of the contact surface with the articular surface. A cut guide(s) is in the body, the cut guide(s) positioned relative to the at least one contact surface so as to be aligned with an underside of the primary implant or of a revision implant.

### CROSS-REFERENCE TO RELATED APPLICATION

The present application claims the priority of U.S. Patent Application No. 63/149,840, filed on Feb. 16, 2021 and incorporated herein by reference.

### FIELD OF THE DISCLOSURE

The present disclosure pertains to tools used for implant revision, such as in knee revision surgery.

### BACKGROUND

An implant revision surgery is a process by which an existing primary implant is removed to be replaced by a new revision implant. Due to the bond between the primary implant and the bone, the bone may often be damaged during implant removal, and the process can be time consuming to limit bone damages. As a result, the subsequent positioning and installation of a revision implant may be complexified due to damaged bone surfaces. For instance, in knee revision surgery, preparation of the bone surfaces using conventional cutting blocks may lack precision as conventional bone landmarks used for defining the orientation of the cutting block may be altered or removed during the removal of the implant.

Accordingly, it has been known to perform a cutting operation by which a saw cuts the bone at the interface with the primary implant, to facilitate the separation of the primary implant from the bone. Such a procedure may be done in a free hand mode by an operator or robot, and may thus result in more bone being removed than needed.

### SUMMARY OF THE DISCLOSURE

It is an aim of the present disclosure to provide a jig for implant revision surgery that addresses issues related to the art.

Therefore, in accordance with a first aspect of the present disclosure, there is provided a jig for revision surgery comprising: a body defining at least one contact surface negatively corresponding to an articular surface of a primary implant, the body configured to be coupled in a unique complementary coupling via engagement of the at least one contact surface with the articular surface; and at least one cut guide in the body, the at least one cut guide positioned relative to the at least one contact surface so as to be aligned with an underside of the primary implant or of a revision implant.

Further in accordance with the first aspect, for example, the body defines a cavity, the at least one contact surface being in the cavity.

Still further in accordance with the first aspect, for example, the at least one contact surface negatively corresponds to the articular surface of a femoral knee implant.

Still further in accordance with the first aspect, for example, the cut guide is located in the body relative to the at least one contact surface to be aligned with a surface of the primary implant or of the revision implant located opposite to a distal cut of the femur.

Still further in accordance with the first aspect, for example, the cut guide is located in the body relative to the at least one contact surface to be aligned with a surface of the primary implant or of the revision implant located opposite to an anterior chamfer cut of the femur.

Still further in accordance with the first aspect, for example, the cut guide is located in the body relative to the at least one contact surface to be aligned with a surface of the primary implant or of the revision implant located opposite to a posterior chamfer cut of the femur.

Still further in accordance with the first aspect, for example, the cut guide is located in the body relative to the at least one contact surface to be aligned with a surface of the primary implant or of the revision implant located opposite to an anterior cut of the femur.

Still further in accordance with the first aspect, for example, the cut guide is located in the body relative to the at least one contact surface to be aligned with a surface of the primary implant or of the revision implant located opposite to a posterior chamfer cut of the femur.

Still further in accordance with the first aspect, for example, the cut guide is a cut slot sized to receive and guide a flat saw blade.

Still further in accordance with the first aspect, for example, the jig has the cut guide on a medial side thereof and/or on a lateral side thereof.

Still further in accordance with the first aspect, for example, the at least one contact surface negatively corresponds to a bone surface of the patient adjacent to the primary implant, the at least one contact surface configured to contact the bone surface.

Still further in accordance with the first aspect, for example, the at least one contact surface negatively corresponds to the articular surface of a tibial knee implant.

Still further in accordance with the first aspect, for example, an assembly comprises the jig as defined above, and a virtual three dimension model of the primary implant in a native state.

Still further in accordance with the first aspect, for example, an assembly comprises the jig as defined above, and the primary implant.

Still further in accordance with the first aspect, for example, an assembly comprises the jig as defined above, and the revision implant.

In accordance with a second aspect of the present disclosure, there is provided a system for generating at least one jig model for implant revision, comprising: one or more processing unit; a non-transitory computer-readable memory communicatively coupled to the processing unit and comprising computer-readable program instructions executable by the processing unit for generating at least one contact surface in the jig model, the at least one contact surface negatively corresponding to an articular surface of a primary implant, for a unique complementary coupling of the at least one contact surface with the articular surface; locating at least one cut guide in the jig model relative to the at least one contact surface, the at least one cut guide aligned with at least part of an underside of the primary implant or of a revision implant; and outputting the jig model.

Further in accordance with the second aspect, for example, the computer-readable program instructions executable by the processing unit are for generating at least one contact surface as negatively corresponding to the articular surface of a femoral knee implant.

Still further in accordance with the second aspect, for example, the computer-readable program instructions executable by the processing unit are for locating the at least one cut guide in the jig model in alignment with a surface of the primary implant or of the revision implant located opposite to a distal cut of the femur.

Still further in accordance with the second aspect, for example, the computer-readable program instructions executable by the processing unit are for locating the at least one cut guide in the jig model in alignment with a surface of the primary implant or of the revision implant located opposite to an anterior chamfer cut of the femur.

Still further in accordance with the second aspect, for example, the computer-readable program instructions executable by the processing unit are for locating the at least one cut guide in the jig model in alignment with a surface of the primary implant or of the revision implant located opposite to a posterior chamfer cut of the femur.

Still further in accordance with the second aspect, for example, the computer-readable program instructions executable by the processing unit are for locating the at least one cut guide in the jig model in alignment with a surface of the primary implant or of the revision implant located opposite to an anterior cut of the femur.

Still further in accordance with the second aspect, for example, the computer-readable program instructions executable by the processing unit are for locating the at least one cut guide in the jig model in alignment with a surface of the primary implant or of the revision implant located opposite to a posterior cut of the femur.

Still further in accordance with the second aspect, for example, the computer-readable program instructions executable by the processing unit are for generating at least one contact surface in the jig model negatively corresponding to a bone surface of the patient adjacent to the primary implant, the at least one contact surface configured to contact the bone surface.

Still further in accordance with the second aspect, for example, the computer-readable program instructions executable by the processing unit are for driving a fabrication of the jig model in 3D printing.

In accordance with a third aspect of the present disclosure, there is provided a system for generating at least one jig model for implant revision, comprising: a contact surface generator module to identify at least one implant abutment surface on a primary implant, and to generate at least one jig contact surface being a negative of the at least one implant abutment surface using a model of the primary implant; and a PSI revision jig generator module to generate and output a jig model using at least the at least one jig contact surface and a geometry of the primary implant or of a revision implant, the jig model including a body defining the at least one jig contact surface and at least one cut guide in the body, the at least one guide positioned relative to the at least one jig contact surface so as to be aligned with a location of an underside of the primary implant or of the revision implant.

Further in accordance with the third aspect, for example, the jig may be included.

Some details associated with the present embodiments are described above and others are described below.

### BRIEF DESCRIPTION OF THE FIGURES

FIG. 1 is a perspective view of a distal end of a femur having a primary implant;

FIG. 2 is a perspective view of a jig for implant revision in accordance with the present disclosure;

FIG. 3 is an exploded perspective view of the jig of FIG. 2 relative to the implant and femur of FIG. 1, prior to coupling for use;

FIG. 4 is a perspective view of the jig of FIG. 2 as uniquely coupled to the implant and femur of FIG. 1;

FIG. 5 is a perspective view of the femur after use of the jig of FIG. 2; and

FIG. 6 is a system for generating at least one patient specific jig model for implant revision.

### DESCRIPTION OF THE EMBODIMENTS

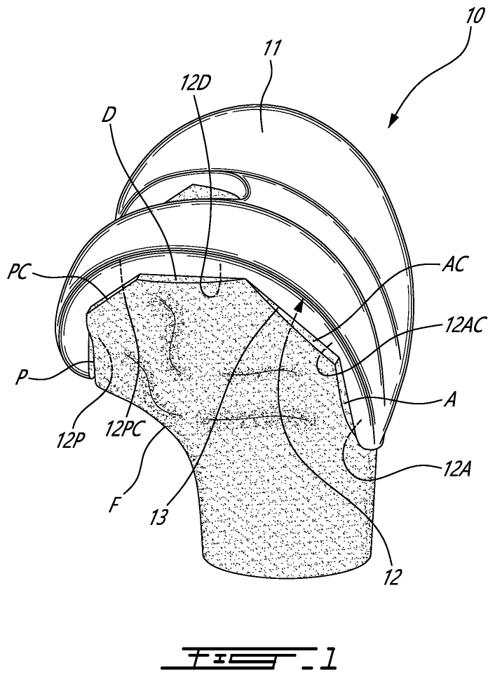

Referring to the drawings, and more particularly to FIG. 1, there is illustrated a distal end of a femur F, with a primary implant 10. The distal femur F is used as an example of a bone that may support a primary implant. Other examples include the tibia (e.g., implant on the tibial plateau), the femoral head, the humerus. The jig of the present disclosure may be used in implant revision for such bones. For simplicity, the present disclosure will describe the jig for knee revision at the distal femur, but may be used with other bones.

The primary implant 10 has an articular surface 11, that is typically worn or damaged due to wear. The implant 10 further has an underside 12 that contacts the bone. In the illustrated embodiment, the underside 12 of the implant 10 is of the type having five planes by which it contacts cut planes of the resected distal femur: underside plane 12A for the anterior cut A, underside plane 12AC for the anterior chamfer cut AC, underside plane 12D for the distal cut D, underside plane 12PC for the posterior chamfer cut PC, and underside plane 12P for the posterior cut P. Other implant geometries may be used for the distal femur, such as geometries that do not feature chamfers, or geometries that do not include an anterior portion, as examples. Moreover, although not seen, one or more pegs may project from the underside 12 of the implant 10 to penetrate corresponding hole(s) in the bone. For example, the peg(s) projects from the underside plane 12D. The expression “underside” is used to express that the underside 12 is concealed, and in contact with the bone, while the articular surface 11 is exposed and in operative engagement with another bone/cartilage (e.g., tibia) or implant on the other bone, such as a tibial plateau implant in the instant embodiment. “Underside” does not relate to the cranial-caudal orientation. The underside 12 may also have surface features, such as a Trabecular Metal™ surface, resulting in an enhanced interface between the bone and the underside 12 of the implant 10, via bone in-growth.

A peripheral edge 13 may be at the junction between the articular surface 11 and the underside 12. The peripheral edge 13 may be continuous. The peripheral edge 13 may therefore have outline segments emulating the shape of the bone cuts, i.e., A, AC, D, PC, P, as observed from FIG. 1.

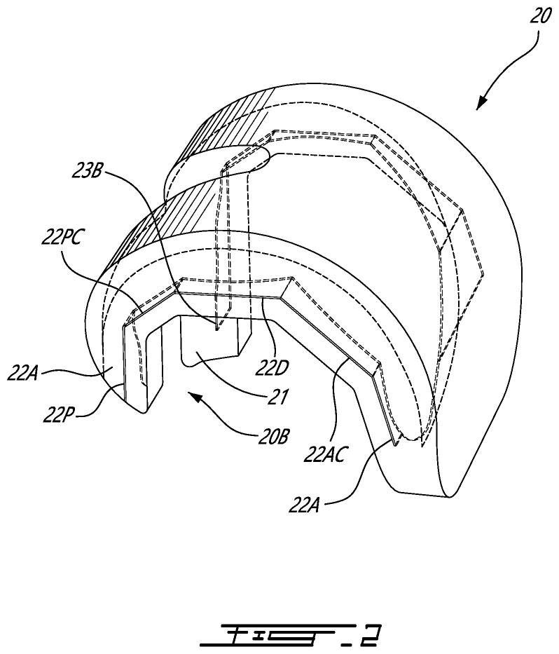

Referring to FIG. 2, a jig for implant revision in accordance with the present disclosure is generally shown at 20. While the expression “jig” is used herein, other expressions could be used to describe item 20, such as a device, an apparatus, a cutting guide, etc. The jig 20 may be said to be implant specific, and/or patient specific. For clarity, reference to implant specific in the present disclosure pertains to the creation of negative corresponding surfaces, i.e., a surface(s) that is(are) the negative opposite of a given surface of an implant, such that the implant specific jig conforms to the implant surface, by complementary confirming contact—the geometry of the jig is specific and unique to the implant, and the implant 10 and jig can only be coupled in a single unique complementary contact manner. The single unique complementary contact manner may be described in a variant as providing a lock or a block, as the complementary surfaces result in a highest level of contact between the implant 10 and jig 20 in comparison to any other coupling between the implant 10 and the jig 20. Jigs as described herein, such as the jig 20, are particularly suited to be used in knee revision in which the tibial knee implant, the femoral knee implant or both implants need to be replaced. The jigs may also be used in other orthopedic implant revision surgery, for instance in shoulder revision surgery. The jig 20 may also be said to be patient-specific, in some embodiments, as it may have a cut slot that is proper to the patient, as described below.

Referring to FIG. 2, the jig 20 has a U-shaped body that is configured to be coupled onto the implant 10 and bone F. In the illustrated embodiment, the jig 20 has a U shape, but other shapes are contemplated, for instance depending on the geometry of the implant with which the jig 20 will be used. The body of the jig 20 is typically made of a rigid material that has limited flexibility, such as a metal or high density polymer. Other materials are contemplated as well. In order to have a unique coupling configuration with the implant, the body of the jig 20 defines a cavity with a contact surface(s) 21 that is a corresponding negative surface of the implant. More particularly, the contact surface 21 is a negative of the articular surface 11, or part of it. For instance, instead of the continuous contact surface 21 shown, the jig 20 may have two or more contact surfaces 21 separated from one another. As the implant tends to be damaged, the contact surface 21 may not be an exact replica of the articular surface 11 in the current state of the implant 10, as wear may not be taken into consideration, and the contact surface 21 may be a negative of the factory state of the implant 10, i.e., undamaged shape, that may be referred to as an unused state, an unimplanted state, a native state. The virtual three-dimensional (3D) model of the implant in this factory state (i.e., as designed and not as used) may be used to create the contact surface(s) 21. The model of the implant may be as shown at 10 in FIG. 1, with or without imaging of the bone.

It is however contemplated to fabricate a jig 20 based on patient-specific imaging, in which a 3D model of the implant 10 is obtained in its current damaged state. This may be done with any appropriate imaging modality, include radiography, computerized tomography, ultrasound, etc. However, when an implant is damaged, it typically has surface material that has worn off whereby the jig 20 may still fit onto the implant 10 in a unique coupling configuration, with the contact surface 21 being the negative of the undamaged geometry of the implant 10. Moreover, a substantial portion of the articular surface 11 of the implant 10 may not be damaged, and this may include the surface of the articular surface 11 surrounding the peripheral edge 13, or a groove (e.g., trochlear groove for femoral implant). Accordingly, in spite of the implant 10 being damaged, a unique coupling configuration may be attained with the jig 20 by the matching surfaces 11 and 21 in the embodiment in which the surface(s) 21 is generated using factory virtual 3D models of the implant 10.

As the jig 20 is implant-specific, the contact surface 21, also referred to as a cavity, may be manufactured with high precision. For example, the jig 20 may be manufactured using the 3D virtual model of the implant 10 as discussed above, or may be cast using a sample of the implant 10. In an embodiment, a virtual 3D model of the implant 10 is provided by the manufacturer. In another embodiment, the contact surface 21 is designed based on specifications of the implant 10. As another possibility, the primary implant 10 and bone may be imaged, and the jig 20 may be patient specific to match the current state of the implant 10. In particular, a combination of radiography and magnetic resonance imagery (MRI) may provide a suitable resolution between implant, bone and cartilage, useful to recognize the boundaries of the implant relative to the bone. The images of the implant may be confirmed, or the model improved using the manufacturer's model of the existing implant. In an embodiment, the method is performed using exclusively radiographic images of the bone and implant. As the jig 20 will abut directly against the primary implant 10 being replaced, radiographic images may provide suitable resolution to assist in the creation of the jig 20. The radiographic images may also assist in performing a surface matching operation to merge the manufacturer's 3D virtual model of the implant with the bone imaging, if desired. For example, the jig 20 may employ techniques as described in U.S. Pat. Nos. 9,924,950, 10,716,579 and 10,881,416 in order to generate a geometry for part or all of the jig 20 using 2D images of a bone and implant, the jig 20 being patient specific. The contact surface(s) 21 may thus abut also with a corresponding bone surface as a result of the imaging, in an embodiment.

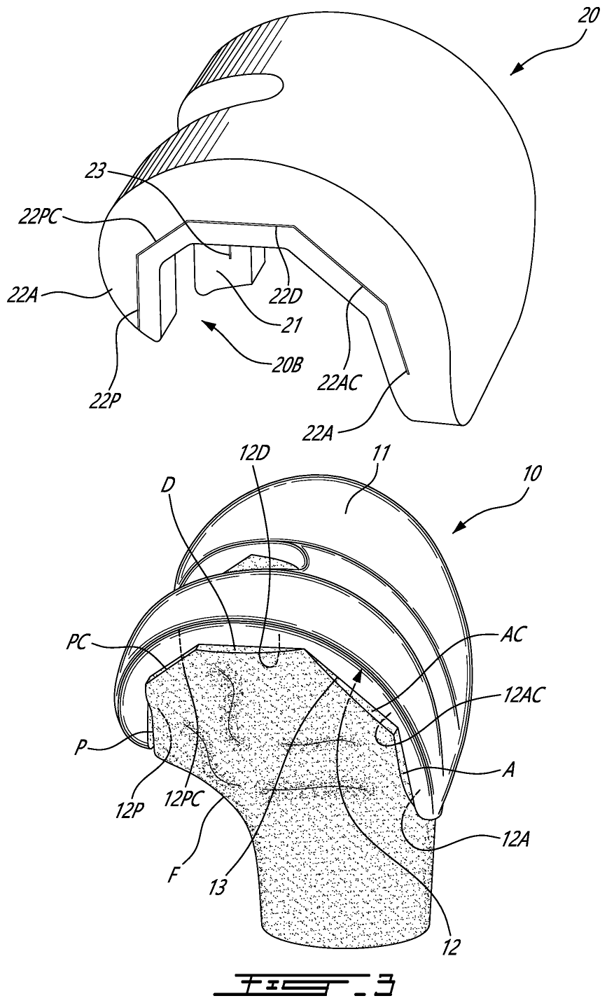

The body may further define an entry way 20B by which the jig 20 may be inserted onto the implant 10 and bone in the direction shown in FIG. 3. The entry way 20B is shown as being arch-shaped, but other shapes are considered, to allow insertion of the implant 10 into the cavity of the jig 20. It is also contemplated to position the jig 20 onto the implant 10 from a lateral or medial direction, whereby the entry way or like clearance would be on a side of the jig 20 to enable this sliding coupling. The body may rely in elastic deformation to be installed onto the implant 10. In such an embodiment, the material used for the jig 20 has such deformation capacity.

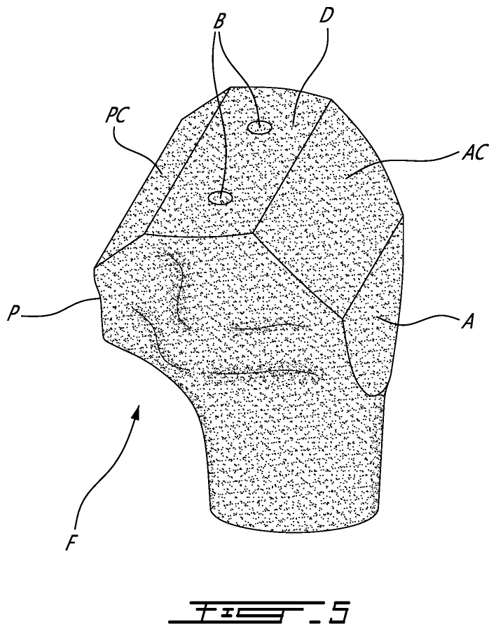

The jig 20 is configured to allow a tool such as a flat sawblade to be cut through the bone at a desired location, so as to separate the implant 10 from the bone. In an embodiment, the jig 20 is configured to pass the sawblade in the bone just below its junction with the implant 10. Therefore, the jig 20 defines a blade slot, generally referred to as 22 and having segments 22A, 22AC, 22D, 22PC and/or 22P (or any single one or any combination thereof) corresponding to the undersurface portions 12A, 12AC, 12D, 12PC and 12P, respectively. In such a case, an outline 23 in the cavity of the jig 20 may generally correspond to the peripheral edge 13 of the implant 10. The blade may therefore be inserted from a lateral or medial position and be aligned with the bone at its junction with the implant 10. The blade may therefore be guided into separating the bone F from the implant 10. As a result, the implant 10 may be removed so as to free up the bone as shown in FIG. 5.

The jig 20 is shown having five cut slot segments based on the type of implant 10 that is used. It is contemplated to have fewer slots based on other types of implants 10. While the slot 22 is shown as being sized for a flat blade, it is also considered to provide a wider slot 22 for other tool heads. Also, while the slot 22 is being shown close ended, it may open as well to an edge of the body of the jig 20. The segments 22A, 22AC, 22D, 22PC and 22P are illustrated as being end to end to form the continuous slot 22, but separate segments are also possible. Likewise, a curved shape could be used instead of the segments shown, to avoid steps, for example if the bone is to be exposed to additional resection after removal of the primary implant. As also shown from FIG. 2, it is possible to have a mirror image of the cut slot segments 22 on the other side of the jig 20. Accordingly, an operator may have two angles of attack. This is merely an option. Stated differently, the jig 20 may have a medial slot 22, and a lateral slot 22.

During use, the jig 20 is mounted onto the implant 10 and bone F in the unique coupling configuration. Although not shown, some mechanisms may be present to secure the jig 20 in position on the implant 10 in the unique coupling configuration, though this may be unnecessary, for instance by the application of suitable force to ensure that the jig 20 remains coupled in the unique coupling configuration with the implant. Fastener holes may be for example present in a portion of the jig 20 aligned with the bone, so as to alternatively secure the jig 20 to the implant 10 via the bone and fastener. In an embodiment, the fastener holes may be placed aligned with segments 22P or 22A, i.e., the first segments likely to be resected, in order to maintain stable contact between the implant 10 and the bone when most (e.g., 4) of the segments of the bone-implant interface will have been resected by the time of the final one or more resections. In an embodiment, the jig 20 is a one-use throwaway component, and may be permanently attached to the implant 10 and be disposed of after use, with the implant 10. A cement or like adhesive, suction forces, may for example be used in such a scenario to ensure that the jig 20 remains secured to the implant 10.

It is understood that some pegs may be present in the implant 10. The saw may not be able to cut through the pegs, but can cut the cortical bone around the pegs to facilitate the release of the implant 10 from the bone F, thereafter exposing the peg holes B.

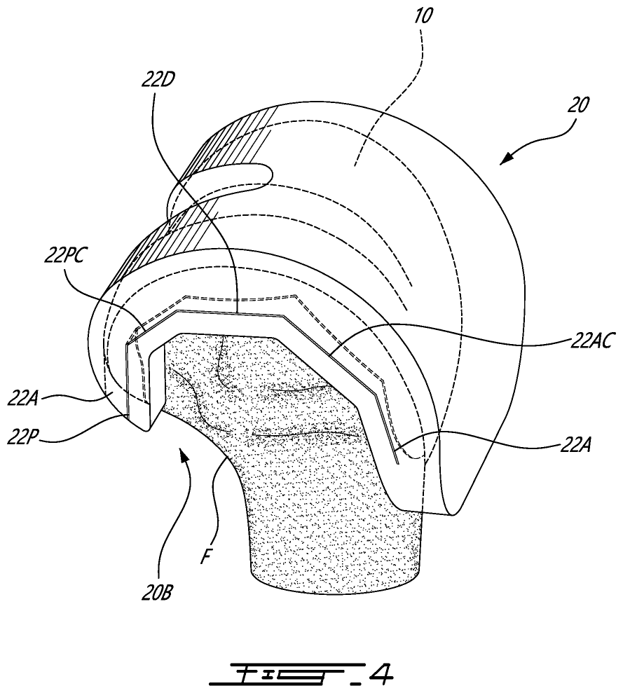

FIGS. 2-4 illustrate the jig 20 as being configured for positioning a saw blade with its slot 22 in close proximity to the junction between the implant 10 and the bone F, so as to minimize the amount of bone removed. In another embodiment, the jig 20 may be configured to position the cut slot 22 in such a way that additional bone may be removed based on the projected revision implant. Indeed, it may be desired to have a different implant that may be slightly thicker than the primary implant. For example, the revision implant may have the same articular surface 11 as the primary implant 10 (or a different one), but may be thicker and therefore have a different underside 12, or smaller surface area. Accordingly, the jig 20 may have a cut slot 22 that does not correspond to the undersurface 12 of the implant 10 that is damaged, but may instead have a cut slot 22 that will reshape the bone F for use with an implant having a different geometry. The cut slot in such a scenario may emulate a peripheral edge of a revision implant. Therefore, in an embodiment, a 3D virtual model may be created to have a contact surface(s) 21 that is a negative of the surface 11 of the implant 10, but may also merge this contact surface(s) 21 with the undersurface of another implant 10, i.e., the implant 10 that will be used as revision implant. In an embodiment, such 3D virtual model may be representative of the revision implant only, for example of the surface 11 of the primary implant and of the revision implant are the same.

Now that jig 20 has been described, a system is set forth for the creation of the jig 20 in accordance with an example of the present disclosure.

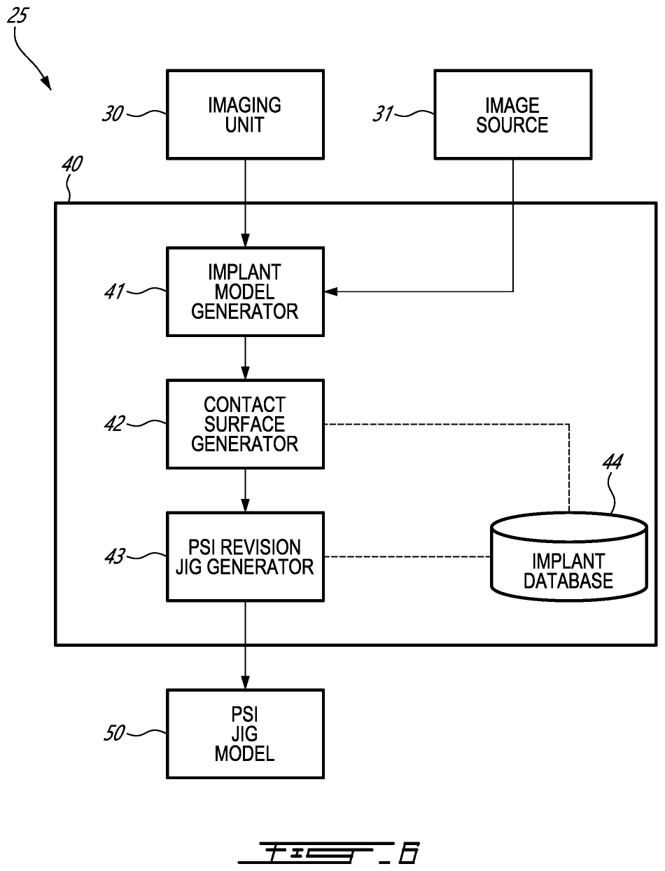

A system for the creation of a jig is generally shown at 25 in FIG. 6. The system 25 may optionally include an imaging unit 30, such as a CT scan or an X-ray machine, so as to obtain images of the bone and implant. As an alternative, images may be obtained from an image source 31, such as obtaining the model of the implant, such as a virtual 3D model as fabricated, as designed, in a native state, in a manufactured state, prior to be being implanted. As an example, a CT scan may be operated remotely from the system 20, whereby the system 20 may simply obtain images and/or processed bone and implant models from the image source 31.

The system 25 comprises a processor unit 40 (e.g., computer, laptop, etc.) that comprises different modules so as to ultimately produce a jig model(s). The processing unit 40 may be used in conjunction with a non-transitory computer-readable memory communicatively coupled to the processing unit and comprising computer-readable program instructions executable by the processing unit 40 for performing steps related to the modules described below.

The processing unit 40 of the system 20 may have an implant model generator 41 receiving images from sources 30 or to generate a 3D model of the jig 20 with its contact surface 21, prior to implant revision. In accordance with the method 10 of FIG. 1, the 3D model of the primary implant 10 may include data pertaining to the surface geometry of a relevant portion of the implant.

The implant model generator 41 will create the 3D model of the implant 10, and may optionally include a part of the bone F. The 3D model of the implant is then used by a contact surface generator module 42 of the processing unit 40. Alternatively, the module 42 may use the 3D model provided by the image source 31, provided the model obtained from the image source 31 includes sufficient data. The virtual 3D model of the implant may be generated using the manufacturer's model of the implant via database 44, and/or via the image source 31 or the imaging unit 30 to account for surface wear and/or via a 3D scan of an identical sample implant.

The contact surface generator module 42 identifies abutment surface(s) on the implant 10 that will serve as support for the jig 20. The abutment surface(s) may thus correspond to the articular surface 11. The contact surface generator module 42 generates a complementary contact surface (or more than one) for the unique coupling configuration described above. The complementary contact surface(s) may correspond to surface 21 in the jig 20, and may be a negative of at least part of the articular surface 11, also known as a contour matching geometry.

Once the jig contact surface(s) has been generated, a PSI revision jig generator module 43 may generate a revision jig model. The reference jig model will have the abutment surface(s) defined to abut against the implant 10, in the unique coupling configuration. As the PSI revision jig 20 will support a saw blade to perform alterations on the bone, the jig model may include cutting planes, guides, slots, or any other tooling interface or tool, oriented and/or positioned to allow bone alterations to be formed in a desired location of the bone, relative to the contact surface(s).

Thus, PSI revision jig generator module 43 may also take into consideration any revision planning done by the operator (e.g., surgeon). The PSI revision jig generator module 43 may also take into consideration a geometry of the revision implant (e.g., obtained from an implant database 44), when the revision implant differs from the primary implant. Therefore, the PSI revision jig generator module 43 combines the contact surface of the primary implant with the geometry of the revision implant, to locate the cut slots 22. The procedure involves a merging of 3D surfaces and geometries, and requires a high level of precision. If the primary implant and the revision implant have the same or a similar articular surface 11, the PSI revision jig generator module 43 may use only the model of the revision implant, as a possibility.

Accordingly, the system 25 outputs PSI jig model(s) 50 that will be used to create the PSI jig 20. The system 20 may also output the PSI jig 20, for example if the system 25 has a 3D printing capability (or any sort, such as stereolithography, laser sintering, FDM, etc) or appropriate machining capability (e.g., CNC). The PSI jig 20 may serve to position cuts in a bone, for instance when a revision implant differs in geometry from a primary implant. The PSI jig 20 may then be used intra-operatively to resurface the bone for subsequent implant installation, with the geometrically different revision implant. It is considered to use the jig 20 as a guide for a robotic arm to cut the planes on the bone.

Therefore, the jig 20 may be generally defined as having a body defining one or more contact surface negatively corresponding to an articular surface of a primary implant for the body to be configured to be coupled in a unique complementary coupling via engagement of the contact surface(s) with the articular surface, and one or more cut guides in the body, the cut guide(s) positioned relative to the at least one contact surface so as to be aligned with an underside of the primary implant, or so as to be aligned with a location of an underside of a revision implant. The alignment may be a coplanar alignment, for instance with the surface of the cut guide(s) being parallel to the undersides, with a slight offset for the cut guide(s) to be opposite bone just adjacent to the primary implant or projected location of the revision implant (e.g., offset of 3 mm or less) An assembly or system featuring the jig, a virtual 3D model of the jig, a virtual 3D model of the primary implant and/or a virtual 3D model of the revision implant may be provided.

The system 25 may generally be defined as being for generating jig model(s) for implant revision, with one or more processing unit and a non-transitory computer-readable memory communicatively coupled to the processing unit and comprising computer-readable program instructions executable by the processing unit. The system 25 may perform functions such asL generating at least one contact surface in the jig model, the at least one contact surface negatively corresponding to an articular surface of a primary implant, for a unique complementary coupling of the at least one contact surface with the articular surface; locating at least one cut guide in the jig model relative to the at least one contact surface, the at least one cut guide aligned with at least part of an underside of the primary implant or of a revision implant; and/or outputting the jig model.

The system 25 may also be defined as being for generating at least one jig model for implant revision. The system 25 may have a contact surface generator module to identify at least one implant abutment surface on a primary implant, and to generate at least one jig contact surface being a negative of the at least one implant abutment surface using a model of the primary implant; and a PSI revision jig generator module to generate and output a jig model using at least the at least one jig contact surface and a geometry of the primary implant or of a revision implant, the jig model including a body defining the at least one jig contact surface and at least one cut guide in the body, the at least one guide positioned relative to the at least one jig contact surface so as to be aligned with a location of an underside of the primary implant or of the revision implant.

While the methods and systems described above have been described and shown with reference to particular steps performed in a particular order, these steps may be combined, subdivided or reordered to form an equivalent method without departing from the teachings of the present disclosure. Accordingly, the order and grouping of the steps is not a limitation of the present disclosure.

1. A jig for revision surgery comprising:
a body defining at least one contact surface negatively corresponding to an articular surface of a primary implant, the body configured to be coupled in a unique complementary coupling via engagement of the at least one contact surface with the articular surface; and
at least one cut guide in the body, the at least one cut guide positioned relative to the at least one contact surface so as to be aligned with an underside of the primary implant or of a revision implant.

2. The jig according to claim 1, wherein the body defines a cavity, the at least one contact surface being in the cavity.

3. The jig according to claim 1, wherein the at least one contact surface negatively corresponds to the articular surface of a femoral knee implant.

4. The jig according to claim 3, wherein the cut guide is located in the body relative to the at least one contact surface to be aligned with a surface of the primary implant or of the revision implant located opposite to a distal cut of the femur.

5. The jig according to claim 3, wherein the cut guide is located in the body relative to the at least one contact surface to be aligned with a surface of the primary implant or of the revision implant located opposite to an anterior chamfer cut of the femur.

6. The jig according to claim 3, wherein the cut guide is located in the body relative to the at least one contact surface to be aligned with a surface of the primary implant or of the revision implant located opposite to a posterior chamfer cut of the femur.

7. The jig according to claim 3, wherein the cut guide is located in the body relative to the at least one contact surface to be aligned with a surface of the primary implant or of the revision implant located opposite to an anterior cut of the femur.

8. The jig according to claim 3, wherein the cut guide is located in the body relative to the at least one contact surface to be aligned with a surface of the primary implant or of the revision implant located opposite to a posterior chamfer cut of the femur.

9. The jig according to claim 1, wherein the cut guide is a cut slot sized to receive and guide a flat saw blade.

10. The jig according to claim 1, wherein the jig has the cut guide on a medial side thereof and/or on a lateral side thereof.

11. The jig according to claim 1, wherein the at least one contact surface negatively corresponds to a bone surface of the patient adjacent to the primary implant, the at least one contact surface configured to contact the bone surface.

12. The jig according to claim 1, wherein the at least one contact surface negatively corresponds to the articular surface of a tibial knee implant.

13. A system for generating at least one jig model for implant revision, comprising:
one or more processing unit;
a non-transitory computer-readable memory communicatively coupled to the processing unit and comprising computer-readable program instructions executable by the processing unit for
generating at least one contact surface in the jig model, the at least one contact surface negatively corresponding to an articular surface of a primary implant, for a unique complementary coupling of the at least one contact surface with the articular surface;
locating at least one cut guide in the jig model relative to the at least one contact surface, the at least one cut guide aligned with at least part of an underside of the primary implant or of a revision implant; and
outputting the jig model.

14. The system according to claim 13, wherein the computer-readable program instructions executable by the processing unit are for generating at least one contact surface as negatively corresponding to the articular surface of a femoral knee implant.

15. The system according to claim 14, wherein the computer-readable program instructions executable by the processing unit are for locating the at least one cut guide in the jig model in alignment with a surface of the primary implant or of the revision implant located opposite to a distal cut of the femur.

16. The system according to claim 14, wherein the computer-readable program instructions executable by the processing unit are for locating the at least one cut guide in the jig model in alignment with a surface of the primary implant or of the revision implant located opposite to an anterior chamfer cut of the femur.

17. The system according to claim 14, wherein the computer-readable program instructions executable by the processing unit are for locating the at least one cut guide in the jig model in alignment with a surface of the primary implant or of the revision implant located opposite to a posterior chamfer cut of the femur.

18. The system according to claim 14, wherein the computer-readable program instructions executable by the processing unit are for locating the at least one cut guide in the jig model in alignment with a surface of the primary implant or of the revision implant located opposite to an anterior cut of the femur.

19. The system according to claim 14, wherein the computer-readable program instructions executable by the processing unit are for locating the at least one cut guide in the jig model in alignment with a surface of the primary implant or of the revision implant located opposite to a posterior cut of the femur.

20. The system according to claim 13, wherein the computer-readable program instructions executable by the processing unit are for generating at least one contact surface in the jig model negatively corresponding to a bone surface of the patient adjacent to the primary implant, the at least one contact surface configured to contact the bone surface.
